(19) United States
(12) Patent Application Publication
(10) Pub. No.: US 2024/0143636 A1
(43) Pub. Date: May 2, 2024

(54) Interactive Chemical Material Interface

(71) Applicant: Ticona LLC, Florence, KY (US)

(72) Inventor: Craig Peterson, Hebron, KY (US)

(21) Appl. No.: 18/481,275

(22) Filed: Oct. 5, 2023

Related U.S. Application Data
(60) Provisional application No. 63/419,358, filed on Oct. 26, 2022.

(51) Int. Cl. G06F 16/332; G06F 16/31; G06F 16/338; G06F 40/205

(52) U.S. Cl. CPC G06F 16/3329; G06F 16/313; G06F 16/338; G06F 40/205

(57) ABSTRACT

Example embodiments of the present disclosure provide for an example method. The example method includes transmitting data packets to cause an interactive user interface to render on a user device. The example method can include obtaining data packets indicative of user input including a query. The query can include features associated with a chemical material or chemical material application. The example method can include parsing an index including a number of chemical materials and associated characteristics. The example method includes obtaining data packets indicative one or more chemical materials that match the features. The example method includes ranking candidate chemical materials based on associated characteristics of each respective candidate chemical material. The example method includes transmitting data packets which cause the user device to display the candidate chemical materials and the associated characteristics for each respective candidate chemical material.

### RELATED APPLICATION

The present application is based upon and claims priority to U.S. Provisional Patent Application Ser. No. 63/419,358, having a filing date of Oct. 26, 2022, which is incorporated herein by reference.

### FIELD

The present disclosure relates generally to exchanging data packets relating to user interfaces associated with interactive chemical material recommendations.

### BACKGROUND

Chemical material recommendation can be a tedious and drawn-out process requiring multiple communications between individuals and consumers. This results in increased processing and potentially duplicative processing of requests from consumers, waiting for responses, and can result in consumers stopping their use of a service. Some existing solutions require users to manually communicate back and forth via online messaging means, telephone calls, sharing documents, etc. A couple disadvantages of the method can be that the method takes much longer and can be susceptible to errors. Moreover, other existing systems can rely on a user first submitting an initial request and manually receiving answers and follow-up questions. This can result in the inefficient exchange of data packets relating to network communications. The existing systems can involve tedious input, which can be inefficient and conducive to errors.

### SUMMARY

Aspects and advantages of embodiments of the present disclosure will be set forth in part in the following description, or may be learned from the description, or may be learned through practice of the embodiments.

One example aspect of the present disclosure is directed to an example computer implemented method. The example method includes transmitting, by one or more computing devices, data to cause an interactive user interface to render via a user device. The example method includes obtaining, by the one or more computing devices, one or more data packets indicative of user input comprising a query. The query includes one or more features associated with a chemical material. The example method includes parsing, in response to obtaining the one or more data packets indicative of the user input, by the one or more computing devices, an index comprising a plurality of chemical materials and associated characteristics. The example method includes obtaining, by the one or more computing devices, from the index, one or more data packets indicative of one or more candidate chemical materials that match the one or more features. The example method includes ranking, by the one or more computing device, the one or more candidate chemical materials based on one or more associated characteristics of each respective candidate chemical material. The example method includes transmitting, by the one or more computing devices, one or more data packets which cause the user device to display the one or more candidate chemical materials and the one or more associated characteristics of each respective candidate chemical material.

One example aspect of the present disclosure is directed to an example computing system, including one or more processors and one or more memory devices storing instructions that are executable to cause the one or more processors to perform operations. In some implementations the one or more memory devices can include one or more non-transitory computer readable media that collectively store instructions, that when executed by the one or more processors, cause the computing system to perform operations. In the example system, the operations include transmitting, by one or more computing devices, one or more data packets to cause an interactive user interface to render via a user device. In the example system, the operations include obtaining, by the one or more computing devices, one or more data packets indicative of user input, wherein the user input comprises a selection of an option to search a document. In the example system, the operations include determining, by the one or more computing devices, based on the user input, one or more features. In the example system, the operations include parsing, in response to obtaining the one or more data packets indicative of the user input, by the one or more computing devices, an index comprising a plurality of chemical materials and associated characteristics. In the example system, the operations include obtaining, by the one or more computing devices, from the index, one or more data packets indicative of one or more candidate chemical materials that match the one or more features. In the example system, the operations include ranking, by the one or more computing device, the one or more candidate chemical materials based on one or more associated characteristics of each respective candidate chemical material. In the example system, the operations include transmitting, by the one or more computing devices, one or more data packets which cause the user device to display the one or more candidate chemical materials and the one or more associated characteristics for each respective candidate chemical material.

In an example aspect, the present disclosure provides for an example non-transitory computer readable medium embodied in a computer-readable storage device and storing instructions that, when executed by a processor, cause the processor to perform operations.

In the example non-transitory computer readable medium, the operations include transmitting, by one or more computing devices, one or more data packets to cause an interactive user interface to render on a user device. In the example non-transitory computer readable medium, the operations include obtaining, by the one or more computing devices, the one or more data packets indicative of user input comprising a query, wherein the query comprises one or more features associated with a chemical material or chemical material application. In the example non-transitory computer readable medium, the operations include parsing, in response to obtaining the one or more data packets indicative of the user input, by the one or more computing devices, an index comprising a plurality of chemical materials and associated characteristics. In the example non-transitory computer readable medium, the operations include obtaining, by the one or more computing devices, one or more data packets indicative of one or more candidate chemical materials that match the one or more features. In the example non-transitory computer readable medium, the operations include ranking, by the one or more computing device, the one or more candidate chemical materials based on one or more associated characteristics of each respective candidate chemical material. In the example non-transitory computer readable medium, the operations include transmitting, by the one or more computing devices, one or more data packets which cause the user device to display the one or more candidate chemical materials and the one or more associated characteristics for each respective candidate chemical material.

### BRIEF DESCRIPTION OF THE DRAWINGS

Detailed discussion of embodiments directed to one of ordinary skill in the art is set forth in the specification, which makes reference to the appended figures, in which:

FIG. 1 depicts a block diagram of an example computing system for generating user interfaces for intelligent recommendations of chemical materials according to example embodiments of the present disclosure;

FIGS. 2A-2C depict an illustration of an example user interface according to example embodiments of the present disclosure;

FIGS. 3A-3C depict an illustration of an example user interface according to example embodiments of the present disclosure;

FIGS. 4A-4F depict an illustration of an example user interface according to example embodiments of the present disclosure;

FIG. 5 depicts an illustration of an example user interface according to example embodiments of the present disclosure;

FIG. 6 depicts an illustration of an example user interface according to example embodiments of the present disclosure;

FIGS. 7A-7B depict an illustration of an example user interface according to example embodiments of the present disclosure;

FIGS. 8A-8E depict an illustration of an example user interface according to example embodiments of the present disclosure;

FIG. 9 depicts an illustration of an example user interface according to example embodiments of the present disclosure;

FIGS. 10A-10C depict an illustration of an example user interface according to example embodiments of the present disclosure;

FIGS. 11A-11C depict an illustration of an example user interface according to example embodiments of the present disclosure;

FIGS. 12A-12C depict an illustration of an example user interface according to example embodiments of the present disclosure;

FIG. 13 depicts an illustration of an example user interface according to example embodiments of the present disclosure;

FIG. 14 depicts an illustration of an example user interface according to example embodiments of the present disclosure;

FIG. 15 depicts an illustration of an example user interface according to example embodiments of the present disclosure;

FIG. 16 depicts a flow chart diagram of an example method to generate and update an interactive chemical material interface according to example embodiments of the present disclosure; and

FIG. 17 depicts a flow chart diagram of an example method to generate and update an interactive chemical material interface according to example embodiments of the present disclosure.

Reference numerals that are repeated across plural figures are intended to identify the same features in various implementations.

### DETAILED DESCRIPTION

Example aspects of the present disclosure are related to exchanging data packets associated with generating and updating interactive chemical material interfaces to intelligently recommend chemical materials based on user input. Recommending chemical materials or other materials to users may be a tedious and drawn-out process that requires multiple communication of data packets between a chemical material distributor and individual consumers. This can include interactions with subject matter experts who can guide consumers on what chemical materials are best suited for the consumer's need (e.g., a specific application). The numerous communications can result in increased processing and potentially duplicative processing of requests from consumers, waiting for responses, and can result in consumers stopping their use of a service. Manual communication between chemical material distributors and individual consumers can require multiple messages, telephone calls, document exchanges, etc. between the respective parties. These communications can be time consuming and susceptible to errors which result in increased processing by existing systems.

Examples of the present disclosure relates to systems and methods for exchanging data packets for generating and updating interactive chemical material interface(s) to intelligently recommend chemical materials based on target features. In some implementations, the interactive chemical material interface can serve as a centralized connecting platform that can be accessed via a user interface. By way of example, a user can provide input including a particular application for which they need a suggested chemical material. The system can parse an index of chemical materials, related applications, and features of the chemical materials for one or more chemical materials that match the requested application (and/or related features). In some instances, the system can determine that more information is needed to provide a recommendation. The system can provide a message to a user indicating one or more follow-up and/or clarification questions. In this way, the system can provide data packets for a guided user experience in aiding the user to present the most relevant recommendations to the user.

In some examples, the system can rank the one or more chemical materials and related chemical material data, e.g., using a machine-learned model, and provide the results for display to a user in a selectable format. The system can optimize the use of space of a user interface (e.g., screen) by providing only the most relevant results. Additionally, or alternatively, the system can provide one or more visual indicators highlighting features of the chemical material that are related to the rank of the chemical material, a percentage match to the search query of the chemical material, etc. The system can obtain data indicative of the user selecting one or more chemical materials. The selection can be to compare one or more chemical materials, select to cause display of a user interface comprising more details about the one or more chemical materials, and/or cause display of a user interface comprising an order form for the one or more chemical materials. In this way, the system can efficiently surface recommendations for chemical materials based on the chemical material's respective features and provide for optimization of the use of the display of the user interface by only surfacing suggestions that are likely to be interacted with by the user (e.g., because they are the best recommendations based on the initial user input).

Aspects of the present disclosure provide for number technical effects and benefits. By intelligently and automatically suggesting recommendations of chemical materials to a user via a user interface, the system can decrease the amount of user input required thus decreasing processing by the system. The system can provide suggestions and refining questions to perform a recommendation process in a matter of minutes or seconds that historically would require several hours or even days of back-and-forth communications between a user (e.g., consumer) and a company representative. Thus, the system reduces bandwidth usage and processing of the system by decreasing the amount of data packets associated with messaging communications exchanged between users (e.g., over the network). Further, aspects of the present disclosure can lead to automation in recommendations and suggestions which can result in reduction of errors due to user input errors, etc. Thus, the present disclosure provides for various technical effects and benefits and improvements to computer-based systems.

With reference now to the Figures, example embodiments of the present disclosure will be discussed in further detail.

### Example Devices and Systems

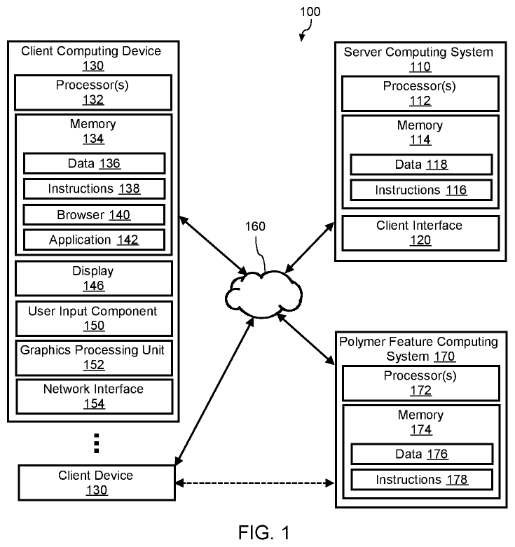

FIG. 1 depicts an example computing system 100 that can be used for generating and updating interactive chemical material interfaces according to aspects of the present disclosure. The system 100 has a client-server architecture that includes a server 110 that communicates with one or more client devices 130 over a network 160. However, the present disclosure can be implemented using other suitable architectures, such as a single computing device unconnected to a network. The computing system 100 can also have a chemical material feature computing system 170 to generate and update interactive chemical material interface(s) based on user input received from a user using the computing system 100.

The computing system 100 includes a server 110, such as, for example, a web server. The server 110 can be one or more computing devices that are implemented as a parallel or distributed computing system. In particular, multiple computing devices can act together as a single server 110. The server 110 can have one or more processor(s) 112 and a memory 114. The server 110 can also include a network interface used to communicate with one or more remote computing devices (e.g., client devices) 130 over a network 160.

The processor(s) 112 can be any suitable processing device, such as a microprocessor, microcontroller, integrated circuit, or other suitable processing device. The memory 114 can include any suitable computing system or media, including, but not limited to, non-transitory computer-readable media, RAM, ROM, hard drives, flash drives, or other memory devices. The memory 114 can store information accessible by processor(s) 112, including instructions 116 that can be executed by processor(s) 112. The instructions 116 can be any set of instructions that when executed by the processor(s) 112, cause the processor(s) 112 to provide desired functionality.

In particular, the instructions 116 can be executed by the processor(s) 112 to implement a client interface 120. The client interface 120 can be configured to provide an interface for a user to provide input indicative of one or more chemical material features. In some implementations, the client interface 120 can be configured to provide instructions to a chemical material feature computing system 170 to generate and update an interactive chemical material interfaced based on user input received from a user using the computing system 100. In particular, in some implementations, the client interface 120 can be provided for interaction with a user using a client computing device 130 that communicates with the server computing system 110 via a network 160.

It will be appreciated that the term “element” can refer to computer logic utilized to provide desired functionality. Thus, any element, function, and/or instructions can be implemented in hardware, application specific circuits, firmware and/or software controlling a general purpose processor. In one implementation, the elements or functions are program code files stored on the storage device, loaded into memory and executed by a processor or can be provided from computer program products, for example computer executable instructions, that are stored in a tangible computer-readable storage medium such as RAM, hard disk or optical or magnetic media.

Memory 114 can also include chemical material data 118 that can be retrieved, manipulated, created, or stored by processor(s) 112. Chemical material data 118 can include one or more characteristics associated with chemical materials and/or one or more chemical material features. For example, chemical material features can include quantitative physical properties, quantitative mechanical properties, quantitative thermal properties, quantitative electrical properties, product features (e.g., flame retardant antioxidant, tribological), automotive specification (e.g., associated with a particular auto manufacturer, material components (e.g., X % glass fiber, X % mineral), processing type (e.g., injection molding, extrusion), delivery form (e.g., pellets, powder), multiple types of requirement (e.g., application and properties), application (e.g., cable glands, 5G connector, cable ties), UL Yellow Card Certification (e.g., V0 at 0.8 mm), competitive grade name and/or competitor TDS, qualitative properties (e.g., stiffness, toughness), regulatory listings (e.g., NSF, WRAS), ASTM call-out (PA specification, PPS specification). As an example, chemical material data 118 can be used to access information and data associated with possible recommended chemical materials based on user input indicative of one or more desired features.

The chemical material data 118 can be stored in one or more databases. The one or more databases can be connected to the server 110 by a high bandwidth LAN or WAN, or can also be connected to server 110 through a network 160. The one or more databases can be split up so that they are located in multiple locales.

The server 110 can exchange data with one or more client devices 130 over the network 160. Although two client computing devices 130 are illustrated in FIG. 1, any number of client devices 130 can be connected to the server 110 over the network 160. The client devices 130 can be any suitable type of computing device, such as a general purpose computer, special purpose computer, navigational device, laptop, desktop, integrated circuit, mobile device, smartphone, tablet, wearable-computing devices, a display with one or more processors coupled thereto and/or embedded therein, or other suitable computing device. Further, a client device 130 can be multiple computing devices acting together to perform operations or computing actions.

Similar to server 110, a client device 130 can include a processor(s) 132 and a memory 134. The memory 134 can store information accessible by processor(s) 132, including instructions that can be executed by processor(s) and data. As an example, memory 134 can store a browser element 140 and an application element 142.

Browser element 140 can provide instructions for implementing a browser. In particular, the user of client device 130 can exchange data with server 110 by using the browser to visit a website accessible at a particular web-address. The chemical material interface(s) of the present disclosure can be provided as an element of a user interface of the website.

Application element 142 can provide instructions for running a specialized application on client device 130. In particular, the specialized application can be used to exchange data with server 110 over the network 160. Application element 142 can include client-device-readable code for providing and implementing aspects of the present disclosure. For example, application element 142 can provide instructions for implementing generation and updating of a chemical material interface user interface application.

The client device 130 can include various user input components 150, or input components, for receiving information from a user, such as a touch screen, touch pad, data entry keys, speakers, mouse, motion sensor, and/or a microphone suitable for voice recognition. In some implementations, the server computing system 110 and the chemical material feature computing system 170 can include their own input components or may share one or more input components with the client device 130, such that a user may be able to provide input for the chemical material feature computing system 170 using the user input component 150 of the client computing device. Further, the client device 130 can have a display 146 for presenting information, such as providing a client interface for obtaining chemical material recommendations. In some implementations, the system can be capable of a plurality of functionalities. For example, the functionalities can include an option to compare candidate chemical materials via the chemical material interface, guide a user's material search, advanced search bar functionalities, gating display of particular individuals based on identity of the user (e.g., a user in an authenticated session), collecting data on user searches to support analytics, generating one or more recommendations, advanced search features (e.g., auto-complete, fuzzy search, auto-correct), consider user data as recommendation input (e.g., user business strategy, past searches, past selections, past orders), uploading TDS, The display 146 can be a visual display including a plurality of visual components for providing the graphical elements of the chemical material interface(s) to a user. The visual display can include a liquid crystal display (LCD), a light-emitting diode display (LED), a plasma display, an organic light-emitting diode display (OLED), and/or a cathode ray tube display (CRT).

The client device 130 can further include a graphics processing unit 152. Graphics processing unit 152 can be used by processor(s) 132 to provide a chemical material interface. In some embodiments, client device 130 performs any and all chemical material interface(s).

The client device 130 can include a network interface 154 for communicating with server 110 over network 160. Network interface 154 can include any components or configuration suitable for communication with server 110 over network 160, including, for example, one or more ports, transmitters, wireless cards, controllers, physical layer components, or other items for communication according to any currently known or future developed communications protocol or technology.

The network 160 can be any type of communications network, such as a local area network (e.g., intranet), wide area network (e.g., Internet), or some combination thereof. The network 160 can also include a direct connection between a client device 130 and the server 110. In general, communication between the server 110 and a client device 130 can be carried via network interface using any type of wired and/or wireless connection, using a variety of communication protocols (e.g., TCP/IP, HTTP), encodings or formats (e.g., HTML, XML), and/or protection schemes (e.g., VPN, secure HTTP, SSL).

In some implementations, the network 160 can be used to transfer data packets between the client computing device 130 and/or the server computing system 110 to the chemical material feature computing system 170. The data packets can include instructions 138 generated in response to receiving a user input via the interactive chemical material interface. For example, the client computing device 130 may receive a plurality of user inputs via the user input components 150 to provide one or more recommended chemical material(s) using the client interface 120. Instructions 138 can then be generated that can be interpreted by the chemical material feature computing system 170 to generate and/or update the chemical material interface(s) to provide one or more chemical material recommendations. Moreover, in some implementations, the instructions 138 can be processed by the one or more processors 172 of the chemical material feature computing system 170 in order to understand the instructions. The chemical material feature computing system 170 can further include memory components 174 for locally storing data 176 and instructions 178. The stored data 176 can include data specific to one or more chemical materials including chemical material features can include quantitative physical properties, product features (e.g., flame retardant antioxidant, tribological), automotive specification (e.g., associated with a particular auto manufacturer, material components (e.g., X % glass fiber, X % mineral), processing type (e.g., injection molding, extrusion), delivery form (e.g., pellets, powder), multiple types of requirement (e.g., application and properties), application (e.g., cable glands, 5G connector, cable ties), UL Yellow Card Certification (e.g., V0 at 0.8 mm), competitive grade name and/or competitor TDS, qualitative properties (e.g., stiffness, toughness), regulatory listings (e.g., NSF, WRAS), ASTM call-out (PA specification, PPS specification). In some implementations, the stored data 176 can include past instructions, information necessary for interpretation, and/or localized models. The stored instructions 178 can include predetermined functions, user-generated functions, and/or trained functions for various tasks the chemical material feature computing system 170 can complete. For example, the stored instructions 178 can include a plurality of instructions for processing user queries obtained via the client interface 120 and generating and/or updating chemical material interfaces based on the user queries obtained via the client interface 120.

In some implementations, one or more non-transitory computer readable media that collectively store instructions that, when executed by the one or more processors, cause the chemical material feature computing system 170 to perform operations can be included in the computing system. In some implementations, the memory components of the client computing device 130, the server computing system 110, and the chemical material feature computing system 170 can include the one or more non-transitory computer readable media. The stored instructions can include instructions that cause one or more of the computing systems to perform operations that can include the methods and processes disclosed herein.

### Example Interface Arrangements

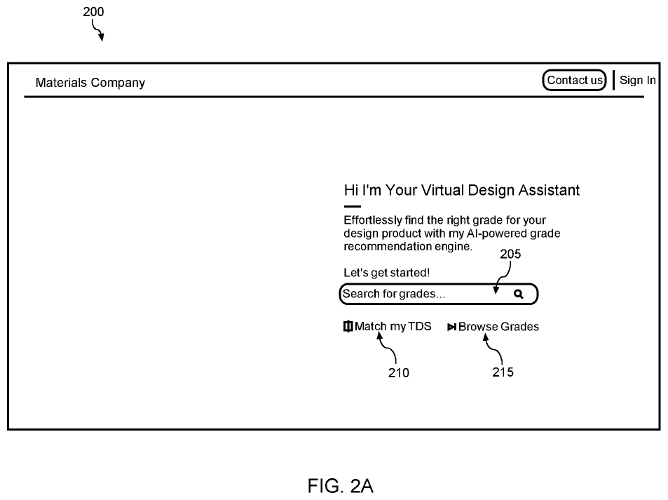

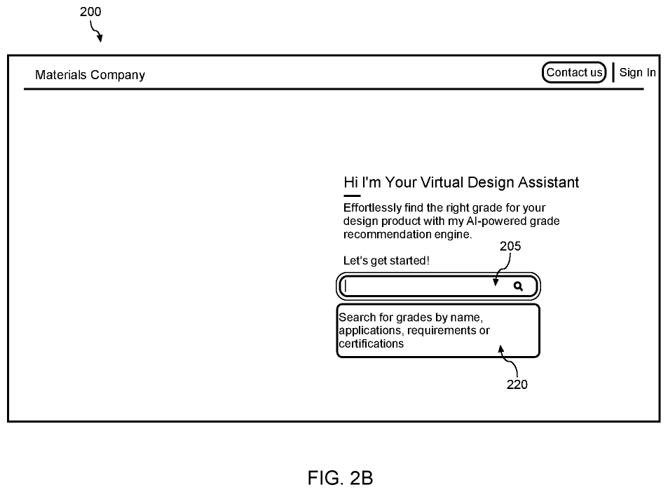

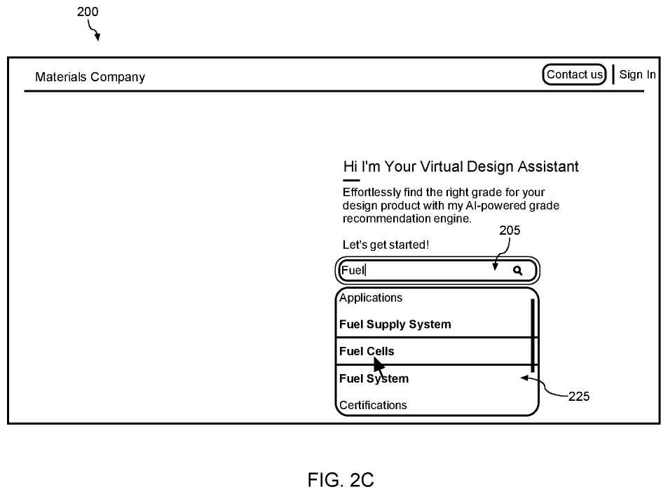

FIGS. 2A-2C depict illustrations of example chemical material interface 200 according to example embodiments of the present disclosure. In some implementations, chemical material interface 200 can include an input field 205.

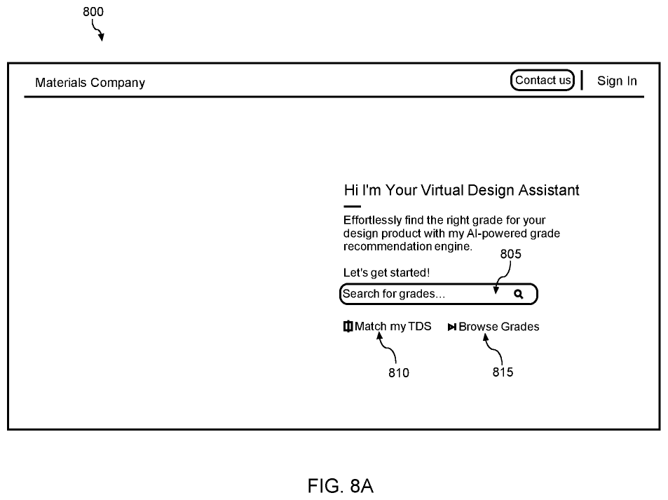

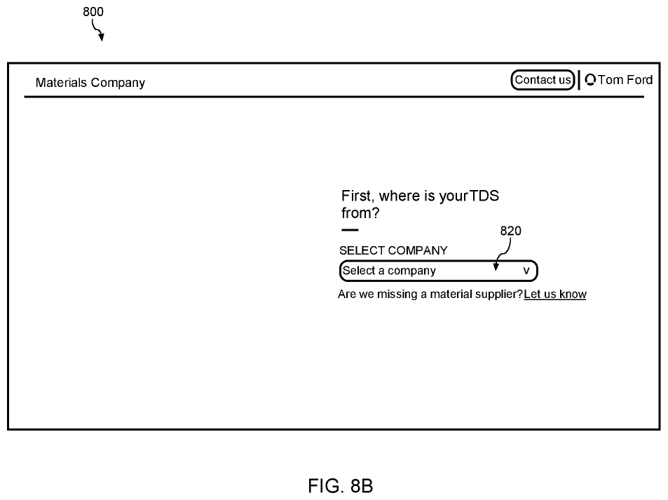

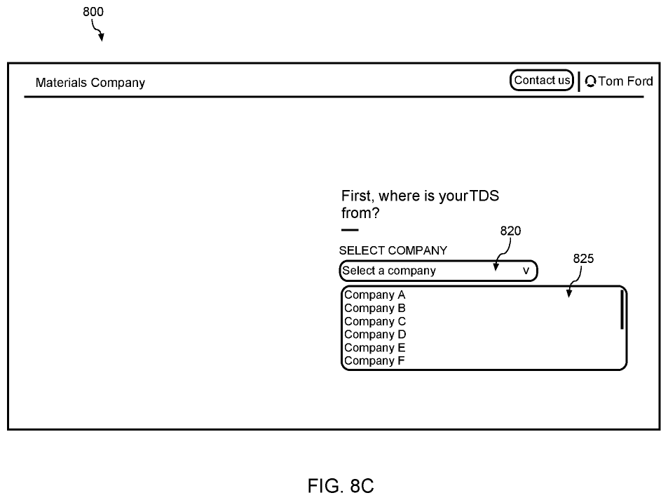

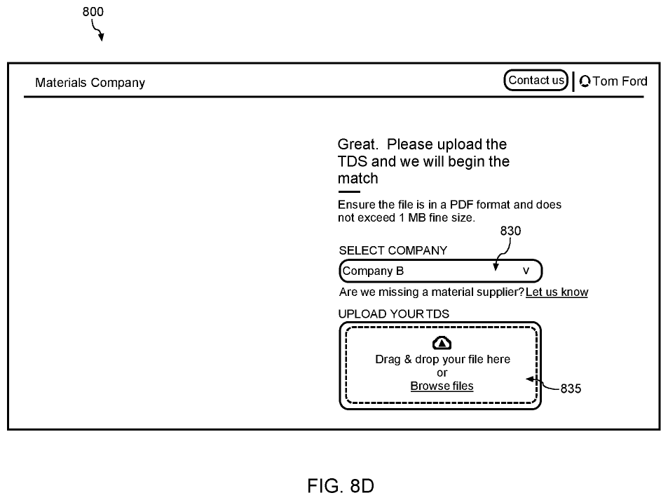

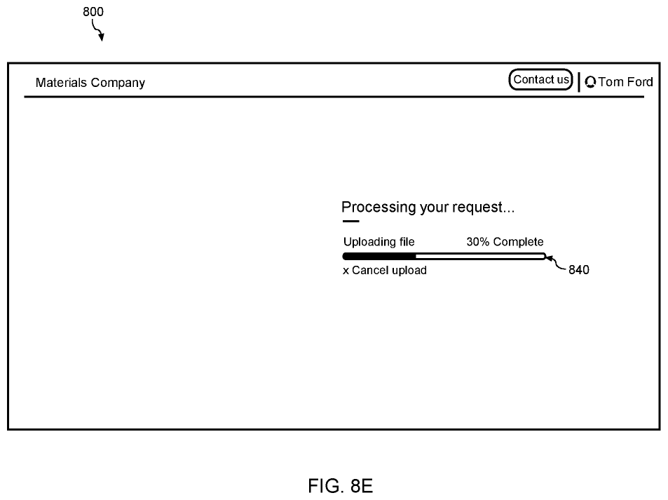

Specifically, FIG. 2A depicts an example chemical material interface 200 before any data packets indicative of user input have been obtained. In this implementation, the chemical material interface 200 can include an input field 205. The field for user input can obtain user input via any reasonable manner, including but not limited to, audio, text, image, etc. The user input can be indicative of a user desire to search for a recommended chemical material. In some implementations, the chemical material interface 200 can include matching a technical data sheet (TDS) element 210, and/or a browsing chemical materials element 215. Matching a technical data sheet (TDS) element 210 and a browsing chemical materials element 215 will be discussed in more detail in the discussion of FIGS. 8A-8E.

FIG. 2B depicts an example chemical material interface 200 including an input field 205 an initial suggestion element 220. For example, the computing system can obtain data packets indicative of user selection of the input field 205. For example, the user can select input field 205 (e.g., through touch screen input, selection via a mouse, audio selection). In response to obtaining the user input, the computing system can provide for display the initial suggestion element 220. In some implementations, initial suggestion element 220 can present a message to a user. For example, chemical material interface 200 can include a message that the user can “search for grades by name, applications, requirements or certifications”. The computing system can obtain data packets indicative of user input via input field 205. In response, the system can provide for an update to example chemical material interface 200 (e.g., as depicted in FIG. 2C).

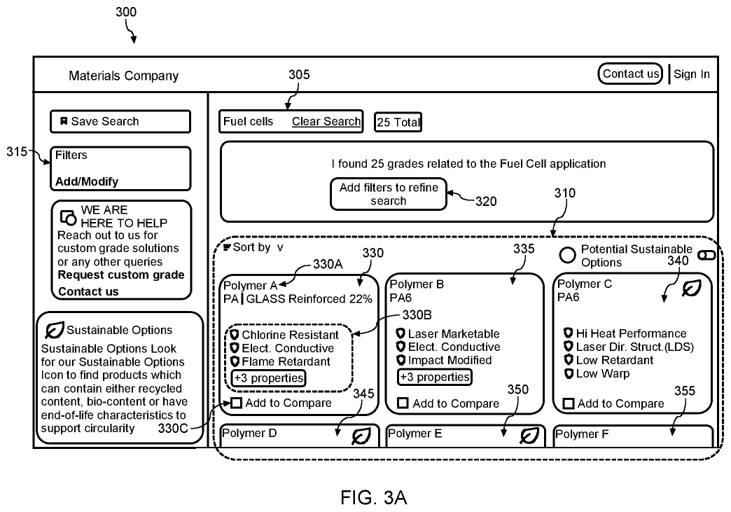

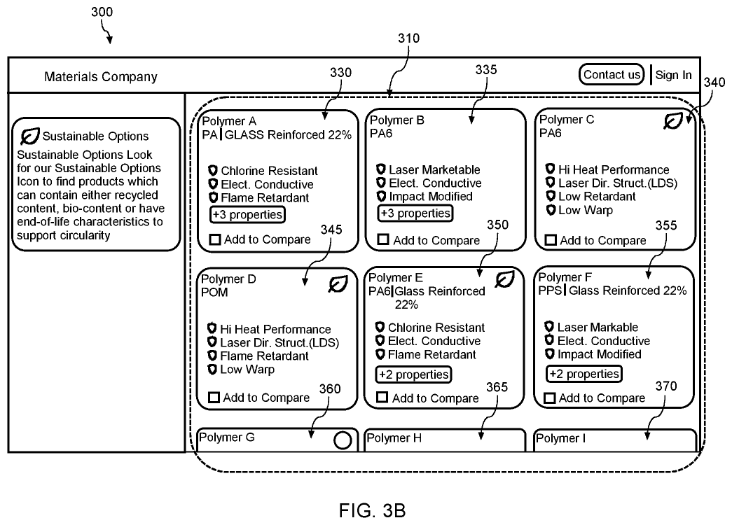

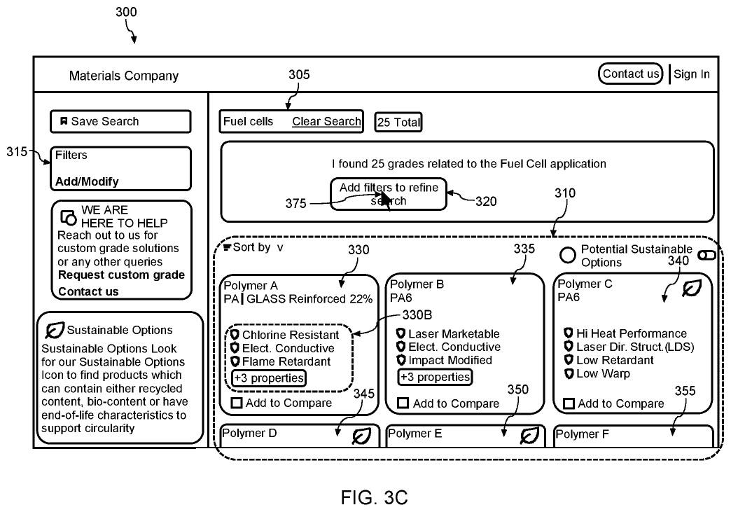

FIG. 2C depicts an example chemical material interface 200 including a field for input field 205 and an updated suggestion element 225. For example, the computing system can obtain data packets indicative of user input via input field 205 indicating a user is searching for “fuel”. In response, the computing system can provide updated suggestion element 225 containing one or more suggestions to guide the user's search. For example, some suggestions can include “Applications” and “Certifications”. As an example, applications can include “fuel supply system,” “fuel cells,” “fuel systems (pumps, filler necks, reservoir) module systems”. The computing system can obtain data indicative of a user selection of one or more suggestion. For example, the user can select “fuel cells”. In response, the computing system can generate and/or update the user interface to provide example a chemical material interface (e.g., chemical material interface 300 as depicts in FIGS. 3A-3C).

FIGS. 3A-3C depict illustrations of example chemical material interface 300 according to example embodiments of the present disclosure. In some implementations, example chemical material interface 300 can include a search input field 305, a search results element 310 (e.g., comprising search results 330, 335, 340, 345, 350, 355, 360, 365, 370), and a filters selectable element 315.

Specifically, FIG. 3A depicts an example chemical material interface 300 depicting example search results according to example embodiments of the present disclosure. For example, the chemical material interface 300 can include a search input field 305. Search input field 305 can include the previously input search (e.g., search input into input field 205). The search input field 305 can also be selected by a user to input an updated search input. Search results element 310 can include a plurality of sub elements (e.g., search results 330, 335, 340, 345, 350, 355). Search result 330 can include a first candidate chemical material (e.g., Chemical material A). Search result 330 can include a chemical material name 330A, one or more features 330B, and a selectable element 330C that can be selected to compare candidate chemical material to one or more additional candidate chemical materials (e.g., associated with search result 335, 340, 345, 350, 355). The search results element 310 can include search result 335, search result 340, search result 345, search result 350, and/or search result 355.

Chemical material interface 300 can include selectable elements to filter the search results. Chemical material interface 300 can include a filters selectable element 315 comprising options for a user to add and/or modify one or more filters. Chemical material interface 300 can include selectable element 320 which can be selected by a user to add filters to refine the user's search. The computing system can obtain packets of data indicative of a user interacting with chemical material interface 300. For example, the user can provide input indicative of a scrolling action and/or a selection of a selectable element and/or input field.

As depicted in FIG. 3B, the computing system can provide further display of chemical material interface 300 in response to obtaining data packets indicative of scroll action of a user. The computing system can provide for display additional search results. The additional search results can be displayed in search result element 310. The additional search results can include search result 360, search result 365, and/or search result 370. As previously described with regard to search result 330 associated with a candidate chemical material, each respective candidate chemical material (e.g., associated with search results 335, 340, 345, 350, 355, 360, 365, 370) can include a chemical material name, one or more features, and a selectable element that can be selected to compare the respective candidate chemical material with one or more additional candidate chemical materials.

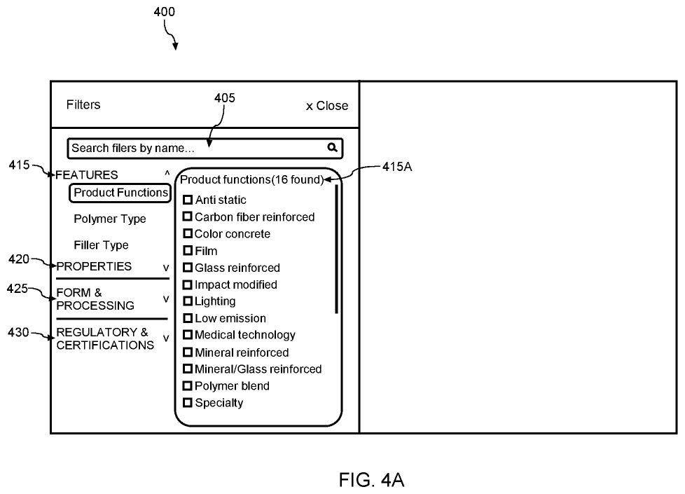

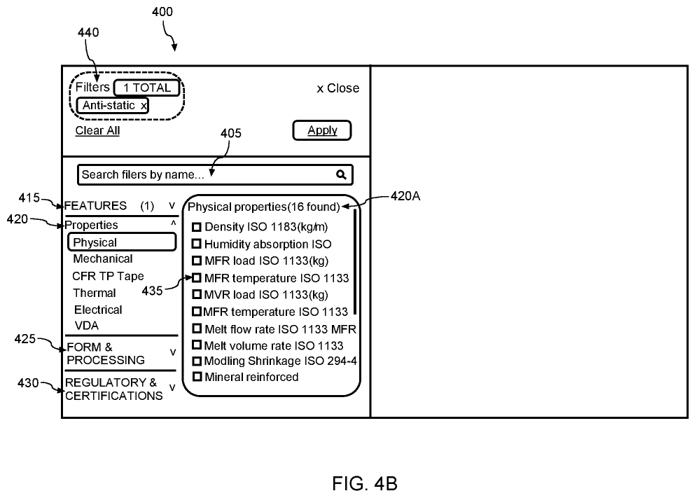

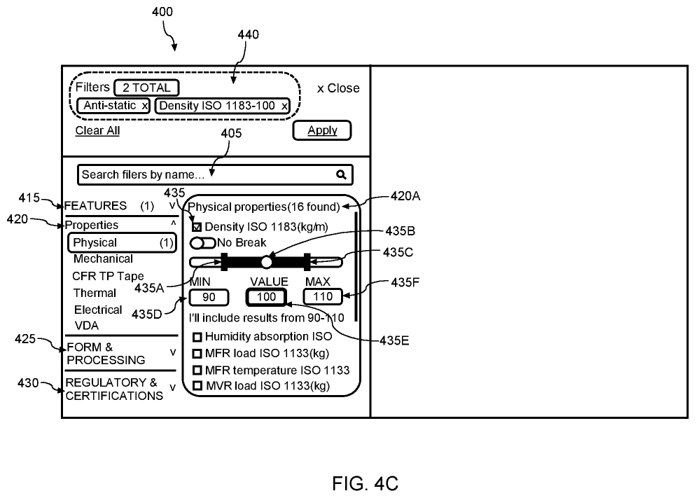

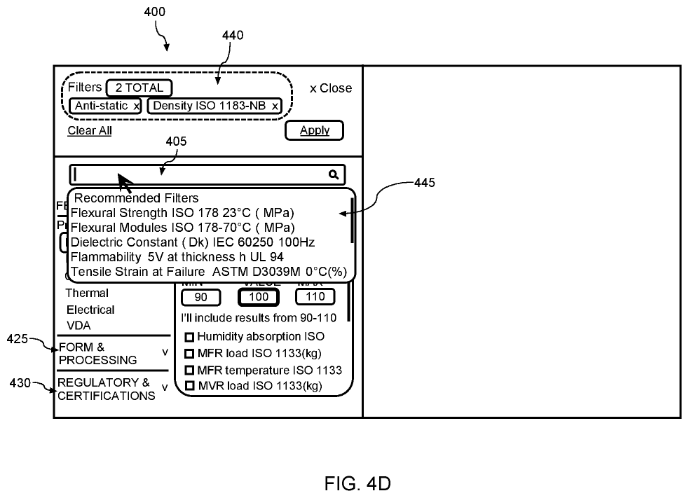

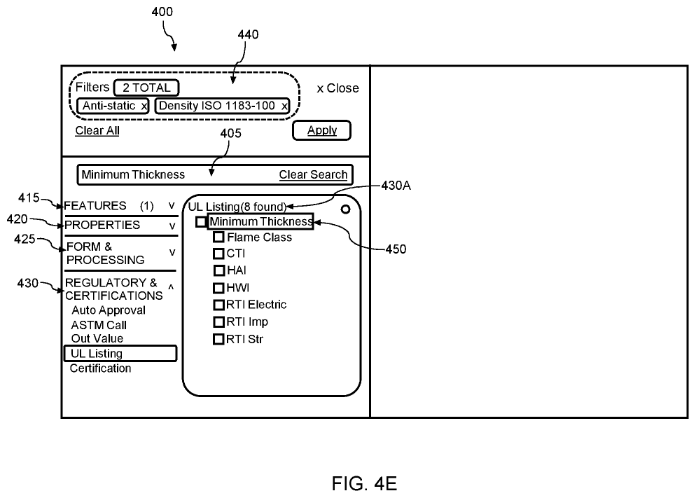

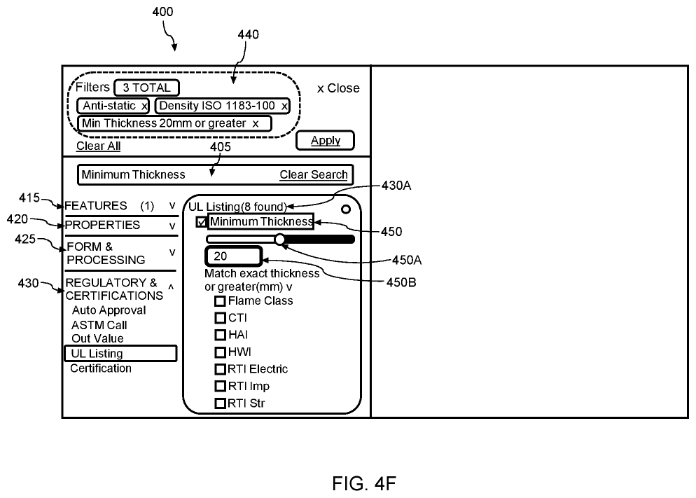

FIG. 3C depicts an example of a user providing input to select the “add filters or refine search” selectable element 320 which can be selected by a user to add filters to refine the user's search. For example, a user can select selectable element 320 using a mouse 375. The computing system can obtain data packets indicative of a user selecting selectable element 320 using mouse 375. In response, the computing system can provide a pop-up filter window (e.g., pop-up filter window 400 depicted in FIGS. 4A-4F).

FIGS. 4A-4F depict illustrations of example pop-up filter window 400 according to example embodiments of the present disclosure. Pop-up filter window 400 can include a plurality of input fields and selectable elements (e.g., search input field 405, and selectable elements 415-430).

As depicted in FIG. 4A, pop-up filter window 400 can include search input field 405. The computing system can obtain data packets indicative of user input indicative of a search for a particular filter in search input field 405. The pop-up filter window 400 can include categories of filters. The categories of filters can include, for example, Features 415, Properties 420, Form and Processing 425, and/or Regulatory and Certifications 430. The computing system can obtain data packets indicative of a user selecting one or more categories of filters. In response, the computing system can provide sub-categories for display. For example, the computing system can cause sub-category 415A along with a plurality of selectable options to be displayed via pop-up filter window 400. For example, the selectable options can include, anti-static, carbon fiber reinforced, color concrete, film, glass reinforced, impact modified, lighting, low emission, medical technology, mineral reinforced, mineral/glass reinforced, chemical material blend, specialty, etc. In some examples, the computing system can obtain data packets indicative of a user providing input indicative of selecting one or more selectable options from sub-category 415A. The user can provide input indicative of selecting another category, for example, properties 420 as depicted in FIG. 4B.

As depicted in FIG. 4B, pop-up filter window 400 can include search input field 405. The pop-up filter window 400 can include categories of filters (e.g., categories 415, 420, 425, 430). The pop-up filter window 400 can include a selected filter element 440 for displaying one or more selected filters. Pop-up filter window 400 can include sub-categories for display. For example, the computing system can cause display of sub-category 420A along with a plurality of selectable options. Sub-category 420A can be associated with physical properties. Selectable options can include, for example, first selectable option 435. First selectable option 435 can be associated with density. Selectable options can include, for example, density, humidity absorption, MFR load, MFR temperature, MVR load, MVR temperature, melt flow rate, melt volume rate, molding shrinkage, mineral reinforced, etc. In some implementations, the computing system can obtain user input indicative of the user selecting first selectable option 435. In response, the computing system can generate and/or provide for display an updated pop-up window (e.g., updated pop-up filter window 400 as depicted in FIG. 4C).

FIG. 4C depicts additional options associated with first selectable option 435. For example, first selectable option 435 can be associated with a filter associated with density. Pop-up filter window 400 can include input for updating a minimum density, maximum density, and desired density. For example, the minimum density 435A, maximum density 435C, and desired density 435B can be input using a sliding bar. Additionally, or alternatively, the minimum density 435D, maximum density 435F, and desired density 435E can be input via input fields (e.g., by entering a numerical value). As depicted in FIG. 4C, the selected filter element 440 can update to display one or more selected filters.

As depicted in FIG. 4D, the system can obtain data packets indicative of a user selecting input element 405. In response, the computing system can provide for display one or more selectable suggested filters in suggestion field 445. For example, the one or more selectable suggested filters can include flexural strength, flexural modulus, dielectric constant, flammability, tensile strain, etc.

FIG. 4E depicts pop-up filter window 400 which can include sub-categories for display. For example, the computing system can cause sub-category 430A along with a plurality of selectable options. Sub-category 430A can be associated with third-party certification listings (e.g., Underwriters Laboratory (UL) listings). Selectable options can include, for example, second selectable option 450. Second selectable option 450 can be associated with minimum thickness. Selectable options can include, for example, minimum thickness, flame class, CTI, HAI, HWI, RTI Electric, RTI Imp, etc.

FIG. 4F depicts additional options associated with second selectable option 450. For example, second selectable option 450 can be associated with a filter associated with minimum thickness. Pop-up filter window 400 can include input for updating a minimum thickness. For example, the minimum thickness 450A can be input using a sliding bar. Additionally, or alternatively, the minimum thickness 450B can be input via an input field (e.g., by entering a numerical value).

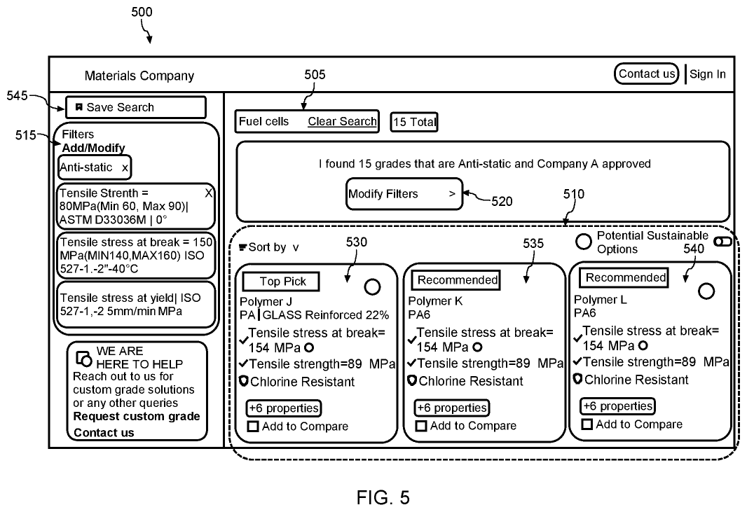

In some implementations, the system can obtain data packets indicative of a user selection of a plurality of filters. In response, the system can generate and/or provide an updated chemical material interface that provides one or more candidate chemical materials and/or a match rating for each respective candidate chemical material as depicted in FIG. 5.

FIG. 5 depicts an illustration of example chemical material interface 500 according to example embodiments of the present disclosure. In some implementations, example chemical material interface 500 can include a search input field 505, a search results element 510 (e.g., comprising search results 530, 535, 540), filters selectable element 515, modify filters selectable element 520, and selectable search element 545.

As depicted in FIG. 5, the chemical material interface 500 can include a search input field 505. Search input field 505 can include the previously input search (e.g., search input into search input field 205, 305). The search input field 505 can also be selected by a user to input an updated search input. Search results element 510 can include a plurality of sub elements (e.g., search results 530, 535, 540). Search result 530 can include a first candidate chemical material (e.g., Chemical material J) that fits specific filter criteria. Search result 530 can include a chemical material name and one or more indications of filters that are associated with the respective candidate chemical material, and a selectable element that can be selected to compare search result 530 to one or more additional candidate chemical materials (e.g., associated with search results 535, 540). The search results element 510 can include search result 535 and/or search result 540.

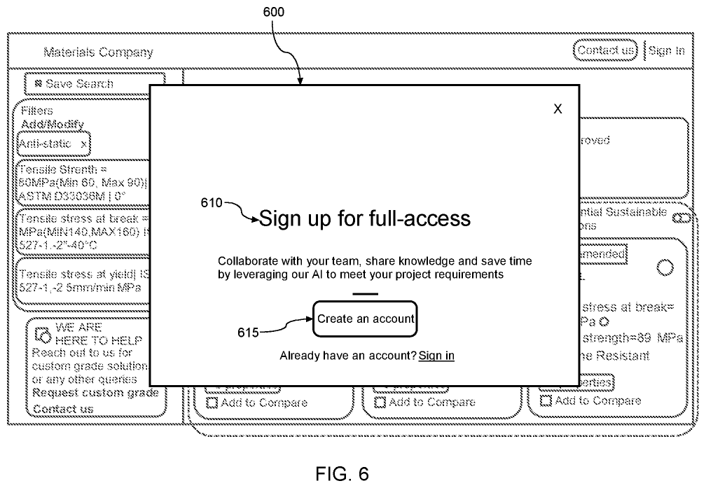

Chemical material interface 500 can include selectable elements to filter the search results. Chemical material interface 500 can include selectable element 515 comprising options for a user to add and/or modify one or more filters. Chemical material interface 500 can include selectable element 520 which can be selected by a user to modify the selected filters. Chemical material interface 500 can include a save search element 545. The computing system can obtain packets of data indicative of a user interacting with chemical material interface 500. For example, the user can provide input indicative of selecting the save search element 545 and/or a particular candidate chemical material (e.g., associated with search result 530). In response to obtaining data packets indicative of a user selecting save search element 545 and/or a particular candidate chemical material (e.g., via selecting search result 530), the computing system can provide pop-up window 600 for display as depicted in FIG. 6.

FIG. 6 depicts illustrations of example pop-up window 600 according to example embodiments of the present disclosure. In some implementations, portions of chemical material interface system can only be accessed by an authenticated user. For example, a save search element 545 feature can only be accessed by users logged into an account. The pop-up window can include a message 610 and a selectable element 615. For example, the message 610 can include a message to a user to “Sign up for full access.” In some implementations the selectable element 615 can be an element that directs a user to a secondary page to create an account associated with the materials company. For example, in response to obtaining data packets indicative of user selecting selectable element 615, the computing system can cause an interface to display which can comprise input fields for a user to supply information to create an account. For example, a user can provide bibliographic data including name, email, phone, etc. Additionally, or alternatively, the computing system can obtain data indicative of a user signing into an existing account.

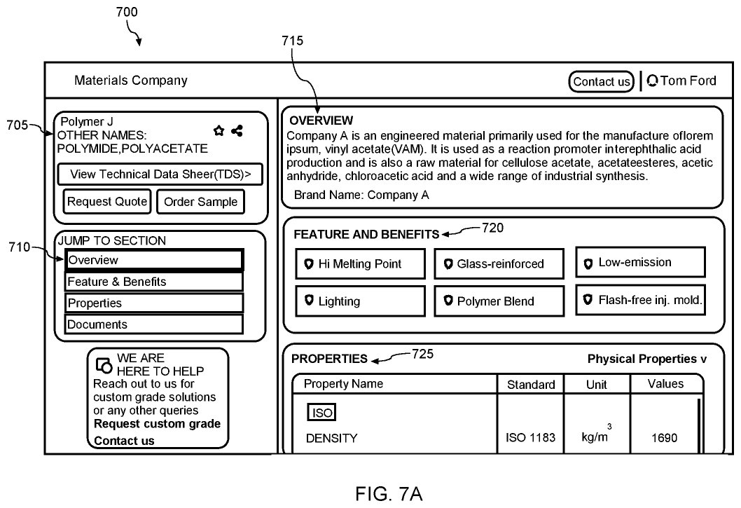

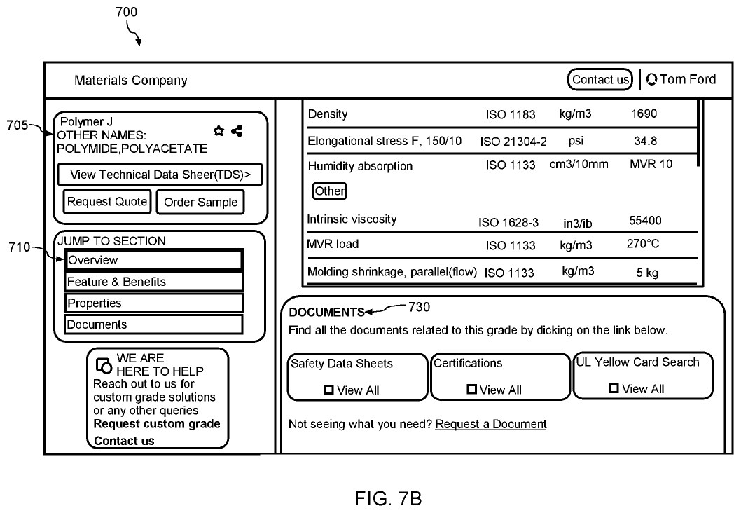

FIGS. 7A-7B depict illustrations of example chemical material interface 700 according to example embodiments of the present disclosure. For example, chemical material interface 700 can include a user interface that is accessible by users in an authenticated session (e.g., by users logged into an account associated with materials company) and associated with a specific candidate chemical material.

More specifically, FIGS. 7A-7B depict a chemical material interface 700 associated with Chemical material J. Chemical material interface 700 can include an introduction element 705, a section summary element 710, an overview section element 715, a features and benefits element 720, a properties element 725, and/or a documents element 730. Introduction element 705 can include a chemical material name (e.g., a brand name), other names the chemical material is referred to as (e.g., generic names, formulaic names, etc.), and/or selectable elements for requesting a quote and/or ordering a sample. Section summary element 710 can include selectable elements that can cause a user interface to update to display specific sections (e.g., section 715, 720, 725, 730). Overview section element 715 can be associated with an overview of the selected chemical material. Features and benefits section 720 can be associated with one or more features and benefits (e.g., associated with one or more search terms, filtered features, etc.). Properties element 725 can include a plurality of properties associated with the selected chemical material. FIG. 7B includes documents element 730. Documents element 730 can include a plurality of selectable elements that can direct a user to one or more documents associated with the selected chemical material. For example, the one or more documents can include safety data sheets, certifications, and/or third-party certifications (e.g., Underwriters Laboratory (UL) Yellow Card Search).

FIGS. 8A-8E depict illustrations of example chemical material interface 800 according to example embodiments of the present disclosure. In some implementations, chemical material interface 800 can include a user input field 805. And a selectable matching technical data sheet (TDS) user input element 820.

Specifically, FIG. 8A depicts an example chemical material interface 800 before any data packets indicative of user input have been obtained. In this implementation, the chemical material interface 800 can include a user input field 805. The field for user input can obtain user input via any reasonable manner, including but not limited to, audio, text, image, etc. The user input can be indicative of a user desire to search for a recommended chemical material. In some implementations, the chemical material interface 800 can include a matching a technical data sheet (TDS) element 810, and/or a browsing chemical materials element 815.

In some implementations, the computing system can obtain data packets indicative of user input indicative of selecting match a technical data sheet (TDS) element 810. In response, the computing system can generate and/or update chemical material interface 800 as depicted in FIG. 8B.

FIG. 8B depicts an example updated chemical material interface 800 that includes a message and an updated user input element 820. Updated user input element 820 can be associated with a plurality of companies that a technical data sheet can be associated with. The computing system can obtain data packets indicative of user selection of user input element 820. In response to obtaining the data packets indicative of the user selection of user input element 820, the system can update chemical material interface 800 to display one or more suggested companies as depicted in FIG. 8C.

FIG. 8C depicts updated user input element 820 and a plurality of selectable suggested companies 825 (e.g., Company A, Company B, Company C, Company D, Company E, Company F, etc.). The computing system can obtain data packets indicative of user selection of one of the suggested companies. For example, the computing system can obtain data packets indicative the user selecting Company B. In response, the computing system can provide an updated chemical material interface 800.

FIG. 8D depicts updated user input element 830 containing the selected company (e.g., Company B) associated with the user input. Chemical material interface 800 can include upload element 835. Upload element 835 can be configured to receive user input. For example, user input can comprise a document uploaded via upload element 835. For example, the user can drag and drop a file to upload and/or browse files (e.g., on a user device) to select for upload. In response to obtaining, the user input comprising the document, the computing system can process and/or analyze the document to extract relevant data. For example, the computing system can perform image recognition, optical character recognition, etc. on the document to extract relevant information.

FIG. 8E depicts an example chemical material interface 800 comprising a screen that indicate that the uploaded document is being process. For example, chemical material interface 800 can include loading bar 840 which can serve as a visual indicator of the processing progress.

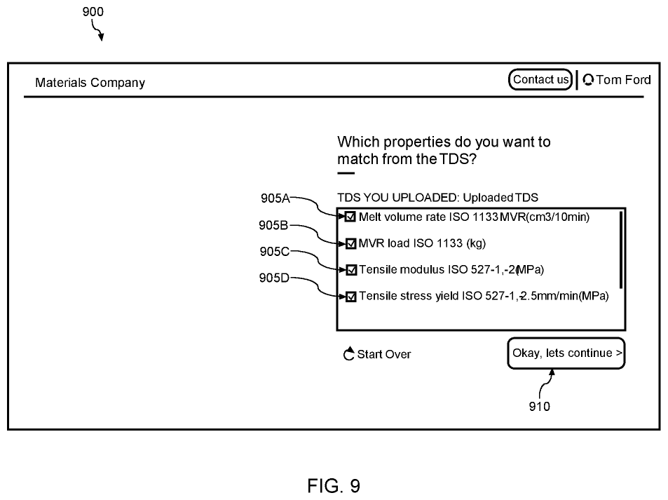

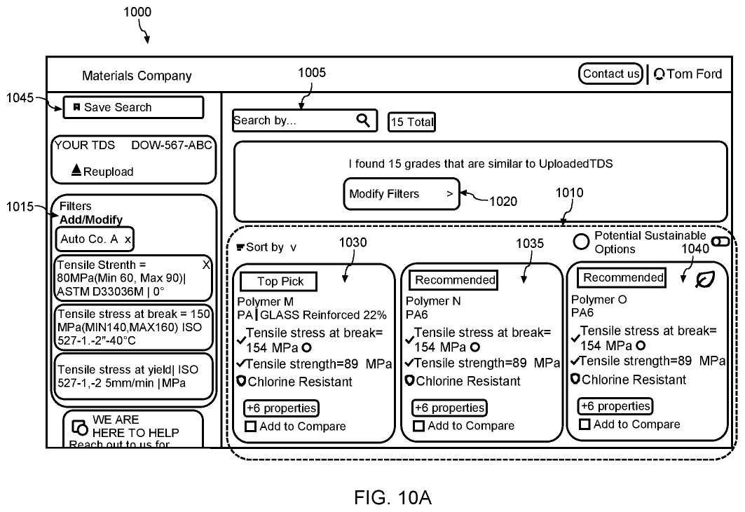

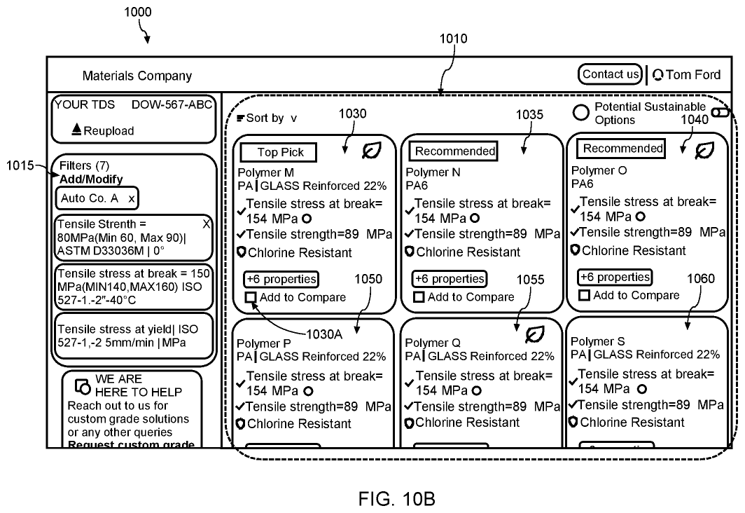

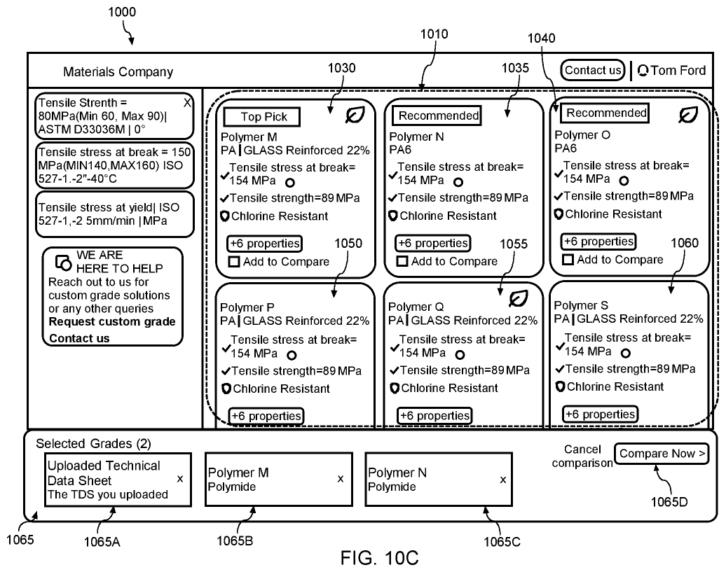

FIG. 9 depicts illustrations of example chemical material interface 900 according to example embodiments of the present disclosure. For example, in response to extracting relevant data from the technical data sheet, the computing system can provide one or more selectable elements associated with one or more extracted properties (e.g., for the system to use as input to find one or more chemical materials with similar properties). For example, the chemical material interface 900 can include selectable component 905A-D. The computing system can obtain data indicative of user selection of a plurality of selectable components (e.g., selectable components 905A-D). The computing system can obtain data indicative of user selection of selectable component 910 indicating that the user would like to proceed to the next user interface. In response, the computing system can generate and provide for display a chemical material interface (e.g., chemical material interface 1000 of FIGS. 10A-C) comprising one or more search results.

FIGS. 10A-C depict illustrations of example chemical material interface 1000 according to example embodiments of the present disclosure.

FIG. 10A depicts an illustration of example chemical material interface 1000 according to example embodiments of the present disclosure. In some implementations, example chemical material interface 1000 can include a search input field 1005, a search results element 1010 (e.g., comprising search results 1030, 1035, 1040), filters element 1015, modify filters element 1020, and/or filters element 1045. For example, the chemical material interface 1000 can include a search input field 1005. Search input field 1005 can include the previously input search (e.g., search input into search input field 205, 305, 505). The search input field 1005 can also be selected by a user to input an updated search input. Search results element 1010 can include a plurality of sub elements (e.g., search results 1030, 1035, 1040, 1050, 1055, 1060). Search result 1030 can include a first candidate chemical material (e.g., chemical material M) that fits specific filter criteria. Search result 1030 can include a chemical material name and one or more indications of filters that are associated with the respective candidate chemical material, and a selectable element 1030A that can be selected to compare search result 1030 to one or more additional candidate chemical materials (e.g., associated with search results 1035, 1040, 1050, 1055, 1060). The search results element 1010 can include search result 1035 and/or search result 1040.

FIG. 10B depicts an example chemical material interface 1000 depicting example search results according to example embodiments of the present disclosure. The computing system can obtain data packets indicative of user selection of an add to compare selectable element (e.g., selectable element 1030A), that can be selected for a plurality of chemical materials to compare.

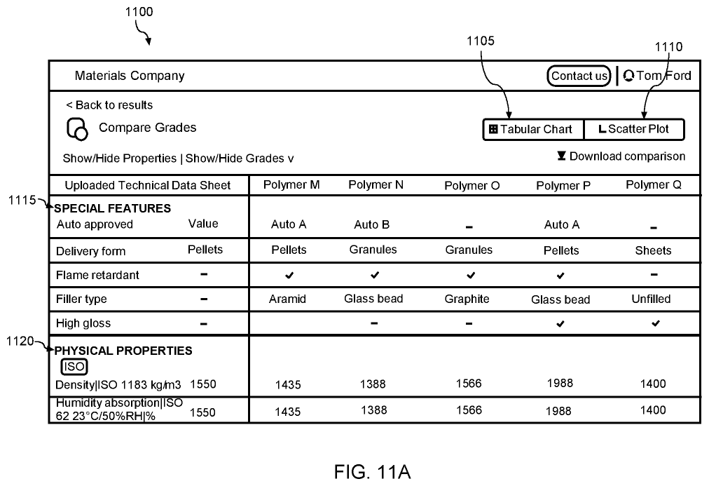

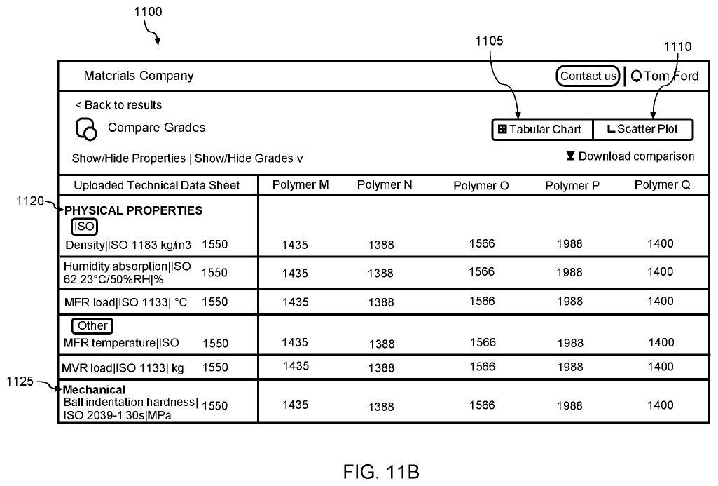

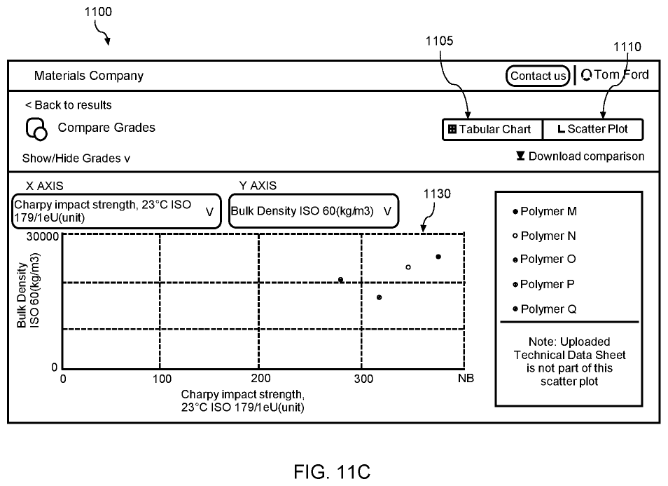

Turning to FIG. 10C. In response to obtaining data packets indicative of user selection of an add to compare selectable element, the computing system can generate pop-up window 1065 comprising a plurality of chemical materials for comparison. For example, the pop-up window 1065 can include an element 1065A associated with the updated technical data sheet, element 1065B associated with candidate chemical material M, element 1065C associated with candidate chemical material N. Pop-up window 1065 can include selectable element 1065D. The computing system can obtain data packets indicative of user selection of selectable element 1065D. In response, the computing system can generate and/or provide for display an updated chemical material interface to display a comparison of the selected candidate chemical materials (e.g., as described with regard to FIG. 11A-D).

FIGS. 11A-11C depict illustrations of example chemical material interface 1100 according to example embodiments of the present disclosure. In some implementations, chemical material interface 1100 can include a selectable element 1105, selectable element 1110, special features element 1115, physical properties element 1120, and/or mechanical properties element 1125.

For example, FIG. 11A can include selectable element 1105 which can be associated with a tabular chart view of the comparison of the features of the candidate chemical materials (e.g., as depicted in FIGS. 11A-11B). Selectable element 1110 can be associated with a scatter plot view of the comparison of the features of the candidate chemical materials (e.g., as depicted in FIG. 11C). Special features element 1115 can include a plurality of special features associated with the candidate chemical materials. For example, special features can include anti-static, chemical resistant, chlorine resistant, cross-linked, electrically conductive, electrostatic dissipation, FDA, flame retardant, filler type, fuel resistant, high crystallinity, high flow, high gloss, high heat performance, high viscosity, hydrolysis resistant, impact modified, improved creep, improved weld line, laser direct structuring (LDS), laser markable, laser transparent, lead-free soldering, light weighting, low emission, low odor, low warp, platable, recycled content, reduced gloss, specialty appearance, sustainable, thermally conductive, tribological, UV resistant, wear resistant, etc. Physical properties element 1120 can include a plurality of physical properties associated with the candidate chemical materials. For example, physical properties can include density, humidity absorption, melt flow rate, melt index, model shrinkage, particle size, molecular weight, etc. As depicted in FIG. 11B, mechanical properties element 1125 can include mechanical properties (e.g., ball indentation hardness, tensile, flexural, impact properties, etc.).

FIG. 11C depicts an example chemical material interface 1100 depicted a scatter plot view of a comparison of a plurality of candidate chemical materials. Chemical material interface 1100 can include scatter plot 1130. In some implementations, the computing system can obtain user input indicative of features to use as the x-axis (e.g., Charpy impact strength) and y-axis (e.g., bulk density).

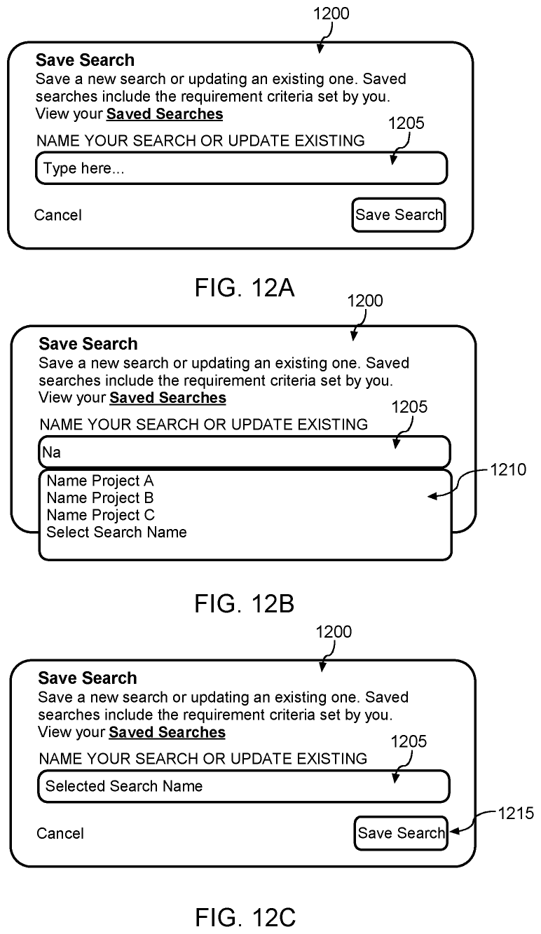

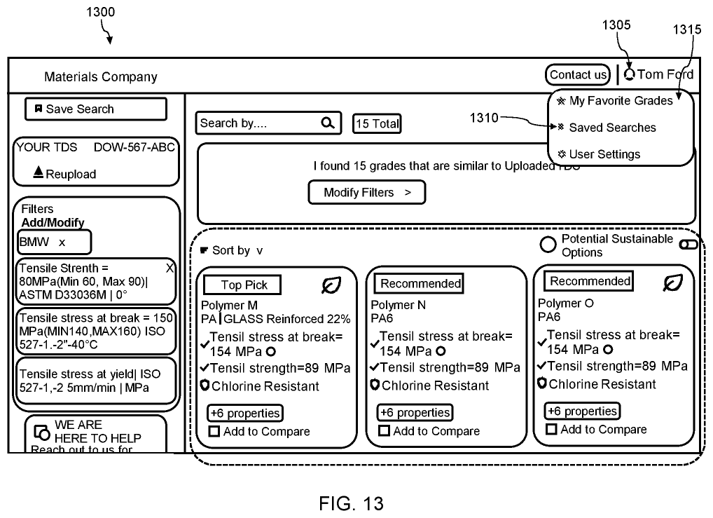

As described in FIG. 5, a user interface can include a save search element 545. The computing system can data packets indicative of user selection of the save search element 545. In response to obtaining the data packets, the computing system can provide an updated chemical material interface as depicted in FIGS. 12A-12C.

FIG. 12A-C depict illustrations of example save search pop-up window 1200 according to example embodiments of the present disclosure. For example, in response to obtaining data indicative of user selection of a save search element 545, the computing system can provide for display save search pop-up window 1200. Save search pop-up window 1200 can include an input field 1205 for obtaining user input indicative of a name to associate with the search (e.g., one or more search inputs, features, filters). The computing system can obtain data packets indicative of user input of a name to save the search. As depicted in FIG. 12B, the save search pop-up window 1200 can be updated to include one or more suggested names 1210 to save the search under. The computing system can obtain data indicative of a user selection of a name to save the search under (e.g., one of the one or more suggested names 1210). The computing system can obtain data packets indicative of user selection of selectable element 1215. In response, the computing system can augment a database with information associated with the present user session (e.g., indicative of search terms, features, filters, etc.) under the selected search name.

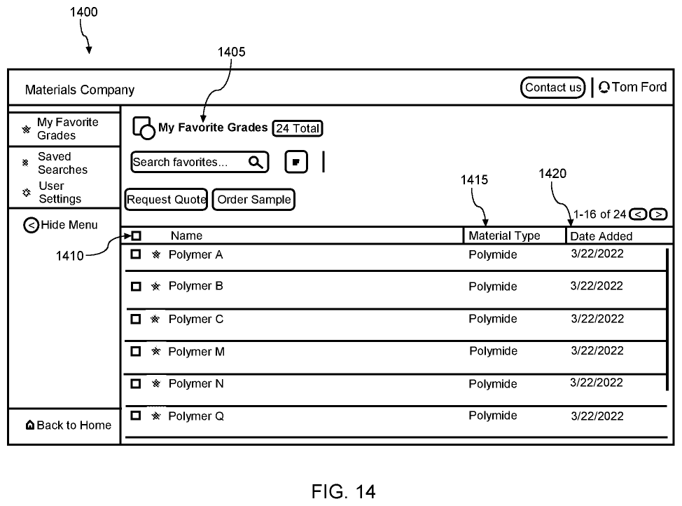

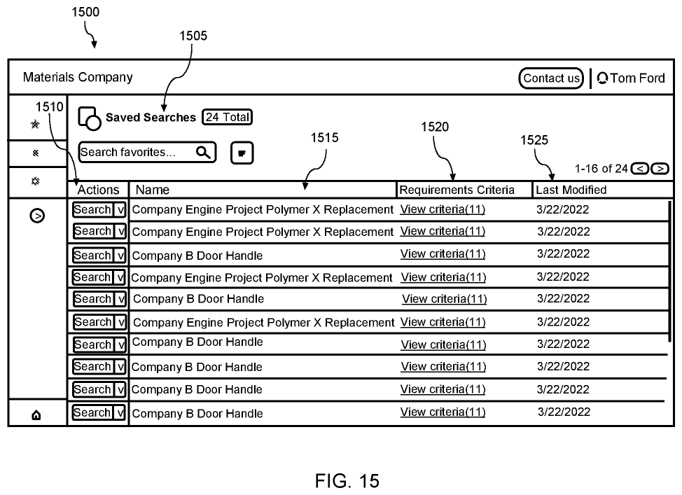

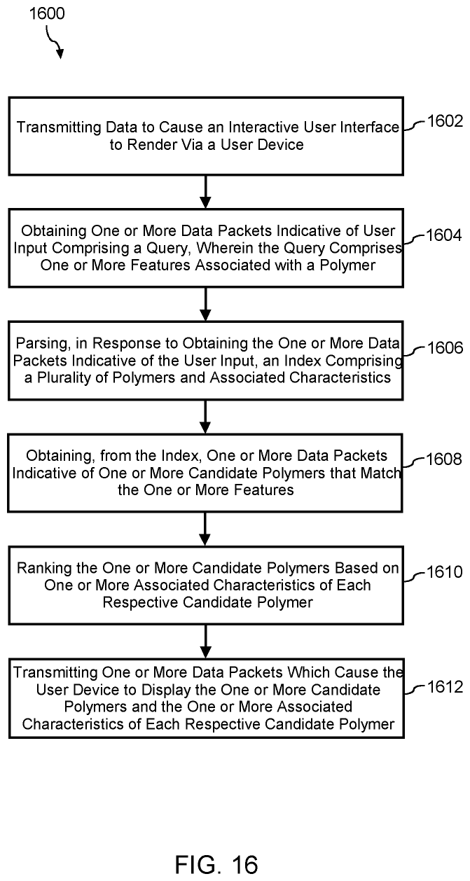

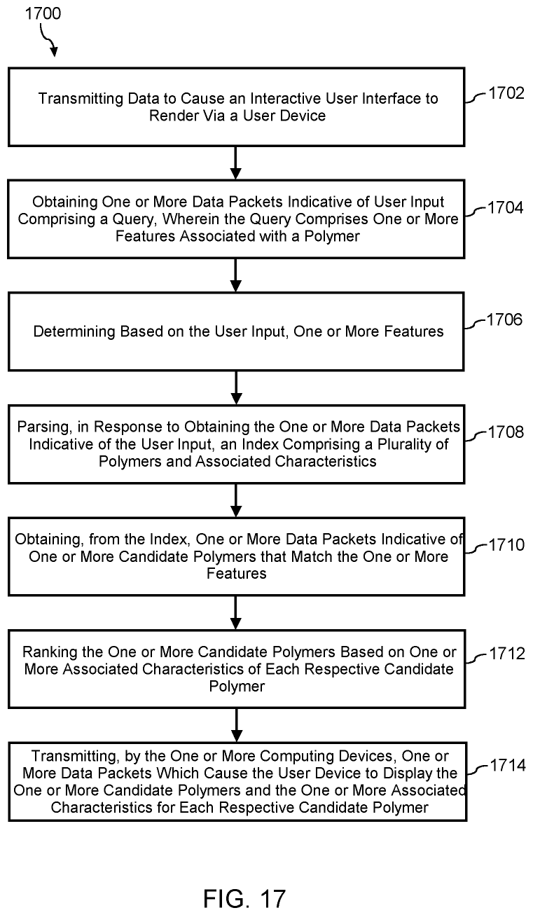

As depicted in FIG. 13, a user can access saved searches via a user account profile. For example, the computing system can obtain data indicative of user selection of an account profile icon 1305. In response, the computing system can update chemical material interface 1300 to provide for display a drop-down menu including a plurality of selectable elements include saved searches element 1310 and my favorite chemical materials element 1315. The computing system can obtain data indicative of user selection of saved searches element 1310 and/or my favorite chemical materials element 1315. In response, computing system can generate and/or provide for display an updated chemical material interface (e.g., chemical material interface 1400 of FIG. 14 and/or chemical material interface 1500 of FIG. 15).

FIG. 14 depicts example chemical material interface 1400. Chemical material interface 1400 can include a title 1405 indicative of the interface indicative of a user's favorite grades. For example, a user can select a selectable element associated with a chemical material to label the chemical material as a “favorite”. In response to obtaining user input indicative of the chemical material being labeled as a “favorite,” the computing system can add the chemical material, label, and/or associated chemical material features to a data store. In response to obtaining user input indicative of selection of a “my favorite grades” selectable element (e.g., favorite chemical materials element 1315), the system can provide for display chemical material interface 1400. Chemical material interface 1400 can include a list 1410 of favorite grades, a column 1415 associated with a respective chemical material's material type, and a column 1420 associated with a respective chemical material's date added.

FIG. 15 depicts example chemical material interface 1500. Chemical material interface 1500 can include a title 1505 indicative of the interface displaying saved searches. In some implementations, the computing system can obtain user input indicative of “saving a search.” In response, the computing system can add the search name, associated features, associated filters, etc. to a data store. In response to obtaining user input indicative of selection of a “saved searches” selectable element (e.g., element 1310), the computing system can provide for display chemical material interface 1500. Chemical material interface 1500 can include a list of saved searches in table form. The table can include an actions column 1510, a name column 1515, requirements criteria column 1520, and last modified date column 1525. Actions column 1510 can include selectable elements. In response to obtaining data indicative of user selection of a selectable element of the table, the computing system can perform an action. For example, the computing system can perform a search comprising the requirements criteria column 1520 of the table.

### Example Methods

FIG. 1600 depicts a flow chart diagram of an example method to perform according to example embodiments of the present disclosure. Although FIG. 1600 depicts steps performed in a particular order for purposes of illustration and discussion, the methods of the present disclosure are not limited to the particularly illustrated order or arrangement. The various steps of the method 1600 can be omitted, rearranged, combined, and/or adapted in various ways without deviating from the scope of the present disclosure.

At (1602), method 1600 can include transmitting data to cause an interactive user interface to render via a user device. For example, a computing system can transmit data to cause an interactive user interface to render via a user device. As described herein, the interactive user interface can be a chemical material interface (e.g., chemical material interface 500, 800).

At (1604), method 1600 can include obtaining one or more data packets indicative of user input comprising query. For example, a computing system can obtain one or more data packets indicative of user input comprising a query. The query can include one or more features associated with a chemical material. By way of example, user input can include at least one of text input, audio input, and/or image input. In some implementations, the input can include a technical data sheet. The technical datasheet can be provided in any file format (e.g., .pdf, WORD, dox, .jpeg, .png, .tiff, etc.). The technical datasheet can be provided through any means (e.g., uploaded, scanned, etc.). For example, the image input can include a scanned copy of a technical data sheet.

In some implementations, input comprises text input. The text input can be processed using natural language processing. The input can include natural language text. The input can include input in a Boolean search format. Natural language processing can be performed using a computer model. For example, the computer model can be a machine learned model.

By way of example, the one or more features can include at least one of (i) a term, (ii) a property, (iii) an application, (iv) a type, (v) a form, (vi) a functionality, or (vii) an industry. For example, a property can include at least one of a chemical property, a physical property, a thermal property, an electrical property or a mechanical property. In some implementations an application can include a specific use. In some embodiments, the industry can include at least one of (i) aerospace, (ii) agriculture, (iii) automotive and transportation, (iv) building and constructions, (v) consumer goods, (vi) electrical and electronics, (vii) food and beverage, (viii) home care and industrial and institutional cleaning, (ix) industrial and manufacturing, (x) medical and pharmaceutical, (xi) oil, gas, and mining, (xii) personal care and cosmetics, and/or (xiii) telecom.

At (1606), method 1600 can include parsing, in response to obtaining the one or more data packets indicative of the user input, an index comprising a plurality of chemical materials and associated characteristics. For example, a computing system can parse, in response to obtaining the one or more data packets indicative of the user input, an index comprising a plurality of chemical materials and associated characteristics. By way of example, associated characteristics can include features. The index can be generate based on obtaining data indicative of historical features of one or more existing chemical materials. For example, the data can be obtained via a user interface.

In some implementations, the plurality of chemical materials comprises at least one polymeric material. By way of example, the at least one polymeric material can include a compounded polymer material comprising one or more thermoplastic polymers and one or more additives.

At (1608), method 1600 can include obtaining one or more data packets indicative of one or more candidate chemical materials that match the one or more features. For example, a computing system can obtain one or more data packets indicative of one or more candidate chemical materials based on one or more features. By way of example, the computing system can obtain data packets indicative of one or more candidate chemical materials that are similar to a current feature. For example, in the past, Grade A can have a successful application. The computing system can obtain data packets, using an artificially intelligent method, comprising an additional grade B and grade C based on one or more similarity to Grade A (e.g., one or more similar features). By way of example, Grade B and Grade C can be selected based on the one or more similarity(ies) to Grade A.

In some implementations, the computing system can determine that the number of candidate chemical materials exceeds a threshold number of candidate chemical materials. The computing system can implement an artificially intelligent method (e.g., using computing models, machine learned models) to generate one or more additional search parameter suggestions to use to narrow the pool of candidate chemical materials. The computing system can provide for display the one or more additional search parameters suggestions in a selectable format via the user interface. The computing system can obtain user input indicative of selection of at least one of the one or more additional search parameter suggestions.

In some implementations, the computing system can determine that the number of candidate chemical materials is below a threshold number of candidate chemical materials. The computing system can implement an artificially intelligent method (e.g., using computing models, machine learned models) to generate one or more suggestions to broaden the number of candidate chemical materials. For example, the computing system can recommend removing one or more filters, updating one or more search terms, etc.

At (1610), method 1600 can include ranking the one or more candidate chemical materials based on one or more associated characteristics of each respective candidate chemical material. For example, a computing system can rank the one or more candidate chemical materials based on one or more associated characteristics of each respective candidate chemical material. By way of example, the ranking can be performed using a machine-learned model. For example, the machine-learned model can be trained using a feedback loop. The training data can include data indicative of user selection of at least one chemical material of the one or more candidate chemical materials. In some implementations, a first search result and associated chemical material and a second search result and associated chemical material can include one or more characteristics that match the one or more features (e.g., of the user input).

The computing system can include a ranking system that provides higher weighting for certain characteristics over other characteristics. By way of example, if a first search result has 10 matching features and a second search result has 10 matching features. The computing system can determine the weight associated with each respective feature to determine a rank for the respective search results. The weights associated with each respective feature can be obtained via user input and/or determined by the computing system. For example, the computing system can obtain data indicative of prior user sessions, in response, the computing system can determine to apply a larger weight to features that are frequently provided via user input by the user associated with the current session. The computing system can also determine weights by obtaining and/or analyzing data associated with a plurality of user sessions associated with a plurality of different users.

At (1612), method 1600 can include transmitting one or more data packets which cause the user device to display the one or more candidate chemical materials and the one or more associated characteristics of each respective candidate chemical material. For example, a computing system can transmit one or more data packets which cause the user device to display the one or more candidate chemical materials and the one or more associated characteristics of each respective candidate chemical material.

In some implementations, method 1600 can include generating, in response to obtaining data indicative of the user input comprising the query, a second interactive user interface comprising a message indicating a follow-up question about the one or more features associated with the chemical material. For example, the computing system can perform one or more natural language processing techniques on the user input. The computing system can us the natural language processing technique(s) to interpret the user input (e.g., indicative of user input obtained via user interface) and, in response, provide for display a follow-up question. Additionally, or alternatively, method 1600 can include obtaining a second user input comprising a response to the message indicating the follow-up question about one or more features associated with the chemical material.

FIG. 1700 depicts a flow chart diagram of an example method to perform according to example embodiments of the present disclosure. Although FIG. 1700 depicts steps performed in a particular order for purposes of illustration and discussion, the methods of the present disclosure are not limited to the particularly illustrated order or arrangement. The various steps of the method 1700 can be omitted, rearranged, combined, and/or adapted in various ways without deviating from the scope of the present disclosure.

At (1702), method 1700 can include transmitting one or more data packets to cause an interactive user interface to render via a user device. For example, a computing system can transmit one or more data packets to cause an interactive user interface to render via a user device. By way of example, the interactive user interface can include a graphical user interface.

At (1704), method 1700 can include obtaining one or more data packets indicative of user input. For example, a computing system can obtain obtaining one or more data packets indicative of user input. The user input can include a selection of an option to search a document. By way of example, the option to search the document can include a search for a document via a document database or a user uploading a document via the interactive user interface. In some implementations, the document can include one or more features associated with a chemical material or chemical material application. In some embodiments, the document can be a technical data sheet of a chemical material.

In some embodiments, method 1700 can include analyzing the document using optical character recognition to obtain one or more features associated with a chemical material or chemical material application. In response to analyzing the document to obtain one or more features associated with the chemical material, generating an interactive user interface comprising a message indicating a follow-up question about the one or more features associated with the chemical material. The method can include obtaining a second user input comprising a response to the message indicating the follow-up question about the one or more features associated with the chemical material.

At (1706), method 1700 can include determining, based on the user input, one or more features. For example, a computing system can determine, based on the user input, one or more features.

At (1708), method 1700 can include parsing, in response to obtaining the one or more data packets indicative of the user input, an index comprising a plurality of chemical materials and associated characteristics. For example, a computing system can obtain the one or more data packets indicative of the user input, an index comprising a plurality of chemical materials and associated characteristics. By way of example, characteristics can be features, filters, etc. associated with chemical materials.

At (1710), method 1700 can include obtaining, from the index, one or more data packets indicative of one or more candidate chemical materials that match the one or more features. For example, a computing system can obtain, from the index, one or more data packets indicative of one or more candidate chemical materials that match the one or more features. By way of example, the method can include determining a match for each respective feature of the plurality of features. In some implementations, the method can include transmitting data that causes display of the match for each respective feature of the plurality of features via the interactive user interface.

At (1712), method 1700 can include ranking the one or more candidate chemical materials based on one or more associated characteristics of each respective candidate chemical material. For example, a computing system can rank the one or more candidate chemical materials based on one or more associated characteristics of each respective candidate chemical material.

At (1714), method 1700 can include transmitting one or more data packets which cause the user device to display the one or more candidate chemical materials and the one or more associated characteristics for each respective candidate chemical material. For example, a computing system can transmit one or more data packets which cause the user device to display the one or more candidate chemical materials and the one or more associated characteristics for each respective candidate chemical material. By way of example, the method can include transmitting, data which causes the interactive user interface to update to display a first selectable user interface element comprising a first chemical material, an overall match rating, a first associated characteristic, and a first match rating associated with the first characteristic.

### Additional Disclosure

The technology discussed herein makes reference to servers, databases, software applications, and other computer-based systems, as well as actions taken, and information sent to and from such systems. The inherent flexibility of computer-based systems allows for a great variety of possible configurations, combinations, and divisions of tasks and functionality between and among components. For instance, processes discussed herein can be implemented using a single device or component or multiple devices or components working in combination. Databases and applications can be implemented on a single system or distributed across multiple systems. Distributed components can operate sequentially or in parallel.

While the present subject matter has been described in detail with respect to various specific example embodiments thereof, each example is provided by way of explanation, not limitation of the disclosure. Those skilled in the art, upon attaining an understanding of the foregoing, can readily produce alterations to, variations of, and equivalents to such embodiments. Accordingly, the subject disclosure does not preclude inclusion of such modifications, variations and/or additions to the present subject matter as would be readily apparent to one of ordinary skill in the art. For instance, features illustrated or described as part of one embodiment can be used with another embodiment to yield a still further embodiment. Thus, it is intended that the present disclosure cover such alterations, variations, and equivalents.

As used herein, the term “chemical materials” may generally encompass polymeric and/or non-polymeric materials. “Non-polymeric” materials may include, for instance, acetyl intermediates (e.g., acetic acid, acetic anhydride, acetic esters, vinyl acetate monomers, etc.), food and beverage ingredients (e.g., acesulfame potassium), preservatives (e.g., potassium sorbate, sorbic acid, sodium benzoate, etc.), and so forth. “Polymeric materials” may include likewise include, for instance, neat polymer systems (e.g., amorphous, semi-crystalline, and/or crystalline polymers), as well as compounded polymer materials containing one or more polymers and one or more additives, such as mineral fillers, fibers (e.g., glass fibers, carbon fibers, etc.), pigments, lubricants, flow modifiers, thermally conductive fillers, electrically conductive fillers, antistatic compounds, and forth. The polymers are typically thermoplastic in nature and may include, for instance, thermotropic liquid crystalline polymers (e.g., LCP), polyarylene sulfides (e.g., polyphenylene sulfide), polyolefins (e.g., propylene homopolymers, propylene copolymers, ethylene polymers, such as ultrahigh molecular weight polyethylene, etc.), aromatic polyesters (e.g., polyethylene terephthalate, polybutylene terephthalate, polycyclohexylenediemethylene terephthalate, etc.), thermoplastic elastomers (e.g., copolyester elastomers, thermoplastic vulcanizates, such as thermoplastic ethylene-propylene-diene terpolymers, etc.), aromatic, semi-aromatic, and/or aliphatic polyamides (e.g., nylon-6, nylon-66, etc.), acetal polymers (e.g., polyoxymethylene), cellulosic derivatives (e.g., cellulose acetate), ethylene vinyl acetate polymers, emulsion polymers (e.g., acrylic polymers), urethane polymers, vinyl fluoride polymers, and so forth.

These and other modifications and variations of the present invention may be practiced by those of ordinary skill in the art, without departing from the spirit and scope of the present invention. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed is:

1. A computer implemented method comprising:
transmitting, by one or more computing devices, data to cause an interactive user interface to render via a user device;
obtaining, by the one or more computing devices, one or more data packets indicative of user input comprising a query, wherein the query comprises one or more features associated with a chemical material;
parsing, in response to obtaining the one or more data packets indicative of the user input, by the one or more computing devices, an index comprising a plurality of chemical materials and associated characteristics;
obtaining, by the one or more computing devices, from the index, one or more data packets indicative of one or more candidate chemical materials that match the one or more features;
ranking, by the one or more computing device, the one or more candidate chemical materials based on one or more associated characteristics of each respective candidate chemical material; and
transmitting, by the one or more computing devices, one or more data packets which cause the user device to display the one or more candidate chemical materials and the one or more associated characteristics of each respective candidate chemical material.

2. The method of claim 1, comprising:
generating, by the one or more computing devices, in response to obtaining the one or more data packets indicative of the user input comprising the query, a second interactive user interface comprising a message indicating a follow-up question about the one or more features associated with the chemical material or chemical material application; and
obtaining, by the one or more computing devices, a second user input comprising a response to the message indicating the follow-up question about the one or more features associated with the chemical material.

3. The method of claim 1, wherein the plurality of chemical materials comprises at least one polymeric material.

4. The method of claim 3, wherein the at least one polymeric material comprises a compounded polymer material comprising one or more thermoplastic polymers and one or more additives.

5. The method of claim 1, wherein the one or more features comprises at least one of (i) a term, (ii) a property, (iii) an application, (iv) a type, (v) a form, (vi) a functionality, or (vii) an industry.

6. The method of claim 5, wherein the property comprises at least one of a chemical property, a physical property, a thermal property, an electrical property or a mechanical property.

7. The method of claim 5, wherein the application comprises a specific use.

8. The method of claim 5, wherein the industry comprises at least one of (i) aerospace, (ii) agriculture, (iii) automotive and transportation, (iv) building and constructions, (v) consumer goods, (vi) electrical and electronics, (vii) food and beverage, (viii) home care and industrial and institutional cleaning, (ix) industrial and manufacturing, (x) medical and pharmaceutical, (xi) oil, gas, and mining, (xii) personal care and cosmetics, or (xiii) telecom.

9. The method of claim 1, wherein the user input comprises at least one of a text input or an audio input.

10. The method of claim 1, wherein the user input comprises an image input, wherein the image input comprises a scanned copy of a technical data sheet.

11. A computing system comprising:
one or more processors;
one or more non-transitory computer readable media that collectively store instructions, that when executed by the one or more processors, cause the computing system to perform operations, the operations comprising:
transmitting, by one or more computing devices, one or more data packets to cause an interactive user interface to render via a user device;
obtaining, by the one or more computing devices, one or more data packets indicative of user input, wherein the user input comprises a selection of an option to search a document;
determining, by the one or more computing devices, based on the user input, one or more features;
parsing, in response to obtaining the one or more data packets indicative of the user input, by the one or more computing devices, an index comprising a plurality of chemical materials and associated characteristics;
obtaining, by the one or more computing devices, from the index, one or more data packets indicative of one or more candidate chemical materials that match the one or more features;
ranking, by the one or more computing device, the one or more candidate chemical materials based on one or more associated characteristics of each respective candidate chemical material; and
transmitting, by the one or more computing devices, one or more data packets which cause the user device to display the one or more candidate chemical materials and the one or more associated characteristics for each respective candidate chemical material.

12. The computing system of claim 11, wherein the option to search the document comprises a search for a document via a document database or a user uploading a document via the interactive user interface.

13. The computing system of claim 11, wherein the document comprises one or more features associated with a chemical material or chemical material application.

14. The computing system of claim 11, wherein the document comprises a technical data sheet of a chemical material.

15. The computing system of claim 11, wherein the operations comprise:
analyzing, by the one or more computing devices, the document using optical character recognition to obtain one or more features associated with a chemical material or chemical material application.

16. The computing system of claim 15, wherein the operations comprise:
generating, by the one or more computing devices, in response to analyzing the document to obtain one or more features associated with the chemical material, an interactive user interface comprising a message indicating a follow-up question about the one or more features associated with the chemical material, wherein the document is analyzed using natural language processing; and
obtaining, by the one or more computing devices, a second user input comprising a response to the message indicating the follow-up question about the one or more features associated with the chemical material.

17. The computing system of claim 11, wherein the matching the one or more candidate chemical materials and respective features comprises:
determining, by the one or more computing devices, a match for each respective feature of the plurality of features; and
transmitting, by the one or more computing devices, one or more data packets that cause display of the match for each respective feature of the plurality of features via the interactive user interface.

18. The computing system of claim 11, wherein the interactive user interface comprises a graphical user interface.

19. The computing system of claim 11, wherein the one or more data packets which cause the user device to display the one or more candidate chemical materials and the one or more associated characteristics for each respective candidate chemical material comprises:
transmitting, by the one or more computing devices, the one or more data packets which cause the interactive user interface to update to display a first selectable user interface element comprising a first chemical material, an overall match rating, a first associated characteristic, and a first match rating associated with the first characteristic.

20. One or more non-transitory computer readable media storing instructions that are executable by one or more processors to perform operations comprising:
transmitting, by one or more computing devices, one or more data packets to cause an interactive user interface to render on a user device;
obtaining, by the one or more computing devices, the one or more data packets indicative of user input comprising a query, wherein the query comprises one or more features associated with a chemical material or chemical material application;
parsing, in response to obtaining the one or more data packets indicative of the user input, by the one or more computing devices, an index comprising a plurality of chemical materials and associated characteristics;
obtaining, by the one or more computing devices, one or more data packets indicative of one or more candidate chemical materials that match the one or more features;
ranking, by the one or more computing device, the one or more candidate chemical materials based on one or more associated characteristics of each respective candidate chemical material; and
transmitting, by the one or more computing devices, one or more data packets which cause the user device to display the one or more candidate chemical materials and the one or more associated characteristics for each respective candidate chemical material.
